(19) United States
(12) Patent Application Publication
(10) Pub. No.: US 2011/0034938 A1
(43) Pub. Date: Feb. 10, 2011

(54) Method for Manufacturing a Device for Positioning and Fixing Electrodes on Body Organs, Device and Kit of Parts

(75) Inventor: Rogier Eric Emile Eijck, Roermond (NL)

Correspondence Address: THE WEBB LAW FIRM, P.C., 700 KOPPERS BUILDING, 436 SEVENTH AVENUE, PITTSBURGH, PA, 15219, US

(73) Assignee: EUROPEAN CUSTOM MANUFACTURING B.V., Gemert (NL)

(21) Appl. No.: 12/864,396

(22) PCT Filed: Jan. 23, 2009
(86) PCT No.: PCT/NL09/50031

§ 371 Date: Oct. 11, 2010

(30) Foreign Application Priority Data
Jan. 23, 2008 (NL) 2001208

(51) Int. Cl. A61B 17/00; H01R 43/16

(52) U.S. Cl. 606/129; 29/874

(57) ABSTRACT

The invention relates to a device for in vivo positioning and fixing of electrodes on body organs, and the manufacture thereof. The invention provides a device for in vivo position, repositioning and fixing of electrodes on body organs, in particular the human heart, comprising: at least one electrode adapted to exchange electrical signals with a body organ, communication means connected to the electrode for co-action with measuring and control means, and at least one resilient fixation element connected to the electrode, wherein the fixation element is expandable from a relatively compact form to a relatively voluminous form, and wherein the fixation element is adapted to press the electrode under bias against the body organ in the relatively voluminous form in co-action with surrounding body tissue in order to maintain the desired position.

The invention relates to a device for in vivo positioning and fixing of electrodes on body organs. The invention also relates to a method for manufacturing the device according to the invention and a kit of parts comprising such a device.

Particularly as a result of improved welfare and ageing of the population there is an increased demand for products for heart stimulation. The purpose of these products is on the one hand mainly focussed on activating the heart muscle in order to guarantee the correct heart rhythm and, if necessary, for a good coordination of the co-action of the two atria and the two ventricles in the heart. The other object thereof is cardioversion, wherein an attempt is made to restore the disrupted rhythm by means of an electric shock.

A second known application is the measuring and controlling of intestinal organs by means of stimulation of the intestinal muscles, wherein the main focus is on the functioning of the contraction and relaxation. Yet another known application where electrodes are used is incontinence, wherein the sphincter muscle is for instance activated/stimulated in order to prevent urine loss.

A device for in vivo positioning and fixing of electrodes on body organs usually comprises for instance an electrode adapted to exchange electrical signals with a body organ and provided with an adhesive material with which the electrode can be adhered at a desired location, and electrical conductors connected to the electrode for co-action with measuring and control means, for supplying electrical pulses to the body organ and/or reading electrical signals from the body organ.

In the current known devices it has firstly been found that it is difficult to fix the electrode at the desired position; the electrode is found to shift during fixing as well as during use. During fixing of the electrode by means of a fixation element the electrode must retain a fixed desired position relative to the organ. If a shift takes place during fixing, it is desirable to reposition the fixation element to the desired position. This repositioning is not possible with the present devices. Repositioning is even desirable in practice, because the surgeon can choose the best positioning and must therefore have the option of being able to reposition. During the period that the electrode must be functionally connected to the body organ, a good fixation of the electrode to the muscles of the body organ is desirable. In addition, it is desirable to introduce the device into the body of the patient, and also be able to remove it again, via a minimal opening in order to minimize the impact on and the risks to the patient.

The present invention has for its object to provide an improved device for in vivo positioning and fixing of electrodes on body organs.

The invention provides for this purpose a device for in vivo positioning, repositioning and fixing of electrodes against body organs, in particular the human heart, comprising: at least one electrode adapted to exchange electrical signals with a body organ, communication means connected to the electrode for co-action with measuring and control means, and at least one resilient fixation element connected to the electrode, wherein the fixation element is expandable from a relatively compact form to a relatively voluminous form and wherein the fixation element is adapted to press the electrode under bias against the body organ in the relatively voluminous form in co-action with surrounding body tissue in order to maintain the desired position. The fixation element is preferably not anchored in a body organ (for instance using barbs) but is conversely fixed against a body organ by pressure, optionally in combination with an adhesive. It is important here that the dimensions of the fixation element are chosen such that in the expanded, voluminous form it is sufficiently large to wedge against the targeted body organ. When the dimensions of the fixation element are too small, a reliable fixing under bias is not possible. The dimensioning depends on the organ for treating and the specific location thereof in the patient. For application in a heart of an adult it is recommended that in the expanded state the fixation element has a size greater than 15 mm×20 mm×5 mm. The fixation element preferably fits into the rectangle with dimensions of 30 mm×50 mm×20 mm, it being generally difficult in the case of larger dimensions to feed the fixation element made from a sufficiently resilient material through a suitable catheter in its compact state. The volume of the fixation element preferably lies between 15 mm×20 mm×5 mm and 30 mm×50 mm×20 mm.

In the compact form such a device is exceptionally easy to introduce into the body and can moreover be positioned particularly easily in the voluminous form, wherein the pressing against surrounding body tissue provides for an improved fixation of the position. During introduction the fixation element can be introduced via a small opening in a compressed state with a relatively small volume. Once the electrode has been brought to the correct position between the targeted body organ and the surrounding body tissue, the electrode can be fixed at the desired position on the body organ by bringing the fixation element into an expanded state, with a relatively large volume relative to the compact state, wherein the electrode and the fixing means are pressed fixedly onto the body organ. If the position must meanwhile be changed, this is possible by temporarily returning the fixation element from its voluminous form to a more compact form. For this purpose the compact form and the voluminous form must preferably be reversible.

The electrode can comprise any electrode known in the prior art or any conceivable electrode suitable for heart and/or intestinal applications. The communication means can comprise electrical conductors, although wireless means such as a radio receiver and/or transmitter can also be envisaged. A typical electrical conductor is manufactured substantially from a metal or metal alloy, for instance substantially from stainless steel. The fixation element can be given an expandable form in different ways, for instance by making use of resilient elements or inflatable elements. The device is preferably manufactured substantially from biocompatible, biodegradable and absorbent materials suitable for surgical applications and preferably provided with an adhesive and homeostatic property. The communication means generally consist of an elongate electrical conductor.

The device according to the invention makes it possible to connect an electrode to a body organ, in particular using a minimally invasive surgical procedure via a catheter. An additional advantage is that the fixation element exerts sufficient pressure in the voluminous state to press for instance fixing means such an adhesive with a determined adhesion time, wherein the relative position of the surfaces for adhering to each other is maintained.

It is advantageous if the fixation element is provided with a profiled surface. The profiled surface has a friction-increasing function. In this way the relative position of the surfaces of the components for connecting can be better maintained during the connecting if a friction-increasing layer is arranged on at least a side of the fixation element remote from the side for adhering. The friction-increasing function is utilized optimally in combination with fixing means such as adhesives.

In a preferred embodiment the electrode is a heart stimulating electrode connected to the fixation element, wherein the fixation element is adapted to press the electrode and fixing means against at least a part of a heart. The device is suitable for application for the heart, wherein the electrode is a heart stimulating electrode and the resilient element is suitable for pressing the electrode and fixing means under bias against at least a part of the heart in co-action with the surrounding tissue. The device according to the invention has the advantage that the good fixation obtained is particularly advantageous for effective measuring and controlling the heart.

In a preferred embodiment the fixation element comprises at least one resilient element. A resilient element makes it particularly easy in the voluminous form to passively exert the desired pressure under bias on the surrounding body organs. A resilient element can for instance be a spring. The fixation element is preferably manufactured substantially from a resilient material. The desired pressure force on surrounding body tissues can be brought about by the dimensioning and design of such a resilient fixation element.

In another preferred embodiment the fixation element comprises at least one expandable body. An expandable body can be used to actively exert pressure on surrounding body tissue. This can for instance be done by supplying a swelling medium to the expandable body or by expanding the material from which the expandable body is manufactured. The expandable body can for instance be a balloon which can be inflated to a voluminous form by a swelling medium, or an absorbent body which can absorb a swelling medium in order to obtain a greater volume. The device preferably comprises a combination of an expandable body and a resilient element.

The expandable body is preferably provided with feed means for feeding a swelling medium to the expandable body. This makes it possible to control the swelling of the expandable body. The feed means can for instance be a feed channel such as a feed hose. The swelling medium can for instance be a gas, a liquid or a foam displaceable by a pump. The feed means can also be used as discharge means if it is desired to have the fixation element transpose from the relatively voluminous form to a more compact form.

It is advantageous if the fixation element is provided with fixing means for adhering the fixation element to the body organ. The fixing means can for instance comprise physical fixing means such as micro-hooks and other fastening means.

In a preferred embodiment the fixing means comprise a biologically acceptable and biologically degradable adhesive. Such adhesives are known in the prior art and are for instance based on materials such as gelatin, collagen and fibrinogen. In combination with a fixation element according to the invention such adhesives have the advantage that by pressing the electrode against the body organ in the relatively voluminous form and in co-action with surrounding body tissue the fixing means can achieve a better and more effective adhesion. It is important here that the fixation element has sufficiently large dimensions that fixing under bias is readily possible and the adhesive can adhere sufficiently firmly. The adhesive preferably comprises at least one electrically conductive component, for instance mobile ions of a salt. An improved electrical conduction is realized by the electrically conductive component. The advantages of this measure are that the production can become cheaper since the electrode surface does not have to remain free of fixing means, and that the physician has the freedom when arranging the electrode to move the electrode a small distance over the body organ without this causing loss of electrical conduction to the body organ. The electrically conductive component can for instance be a physiologically acceptable electrolyte, in particular organic and inorganic salts such as sodium chloride and/or potassium chloride.

It is recommended that the adhesive is an adhesive which can be activated. Such an adhesive is not yet active during introduction of the electrode so as to allow the introduction to progress effectively without the undesirable occurrence of adhesion or contamination of surgical instruments such as a catheter, or body organs with which the device may come into contact during the introduction. Once the electrode has been placed at the desired position the actuable adhesive is activated, after which the device adheres to the body organ. Another advantage of such fixing means is that the resilient element can still be displaced before activation of the adhesive in order to obtain the desired position of the electrode relative to the body organ without this adversely affecting the connecting strength or the body organ being damaged hereby. The activation can for instance be chemical, through the addition of a catalyst or reagent with which the adhesion process of the adhesive is started or accelerated. Physical activation by for instance infrared or UV radiation can also be envisaged. Such adhesives are commercially available.

In a preferred embodiment the actuable adhesive can be activated by mechanical pressure. Pressure-activated medical fixing means are per se known and commercially available under the brand name ‘Gelita sponge’ and ‘Tacosil’, among others. Such an adhesive must be held under pressure for a time to enable activation thereof. Only then does a good connection occur, both in terms of positioning and connection strength. Such a pressure-activated adhesive makes it particularly easy to realize a fixing with the adhesive under the pressure exerted by the fixation element in the relatively voluminous state.

In another embodiment at least a part of the fixing means is protected by a releasable film. This enables a better conditioning of the fixing means during storage of the device. The fixing means will cure less easily under the influence of the ambient air and the device will be less likely to adhere to surrounding surfaces during storage. If it is desired to keep the film on the layer of fixing means during introduction, it is then only necessary to remove the film using suitable surgical instruments when the resilient element has the correct position for adhesion to the body organ. The film is preferably made from a polymer, for instance a suitable nylon (polyamide) or Teflon® (polytetrafluoroethylene).

It is advantageous if the electrode is connected releasably to the fixation element. This makes it possible to remove the electrode from the body in simple manner and without damaging the body organ. The fixation element can then remain in the body or be removed separately, preferably in compressed state.

In a preferred embodiment the electrode is connected to the fixation element such that the mechanical resistance for releasing the electrode from the fixation element is lower than the adhesion resistance between the fixation element and the body organ. It is thus easy to detach the electrode without an extra stabilization of the fixation element here being necessary. This can for instance be realized in that the coupling between the fixation element and the electrode is provided with a weakened portion. The adhesion resistance can comprise, among others, the frictional resistance between the fixation element and the body organs, in addition to additional resistance due to optionally used fixing means such as an adhesive.

It is advantageous if the fixation element is manufactured substantially from a biodegradable material. This has the advantage that, after the at least one electrode has been detached from the resilient element, the fixation element is degraded and/or discharged by the human body without noticeably adverse side-effects and without damage to the body organ for measuring and controlling. Biodegradable materials can for instance be based on gelatin, collagen, lactic acid and/or polysaccharides.

In a preferred embodiment biodegradable material comprises at least one protein selected from the group consisting of gelatin, collagen and fibrinogen. Such biodegradable materials are particularly suitable. Derivatives of such biodegradable materials are also particularly suitable.

It is advantageous if the biodegradable material comprises at least one polysaccharide selected from the group consisting of cellulose, chitin, chitosan and carrageenan. These biodegradable materials contribute toward a good adhesion of the fixation element.

In a preferred embodiment the biodegradable material forms a resilient sponge structure. A sponge structure is resilient and compressible, whereby a transition between the compact form and the voluminous form can also be realized in simple manner. Sponge-like materials can for instance be manufactured on the basis of gelatin, fibrinogen, collagen, chitin or cellulose.

The biodegradable material is preferably formed substantially from sponge-like collagen. Sponge-like collagen is a particularly suitable material which has also been found to have good mechanical properties for use in the body.

It is advantageous if the fixation element is at least partially provided with a reinforced cover layer. A cover layer helps to protect the body organs and contributes toward the distribution of forces between the fixation element, body organ and surrounding body tissue. The cover layer is preferably formed substantially from a biologically acceptable polymer material, which is preferably fibre-reinforced. Suitable materials can be based on for instance gelatin, fibrinogen, collagen, chitin or cellulose. A particularly suitable material for the cover layer is reinforced collagen. In another preferred embodiment the cover layer is manufactured from a woven material preferably manufactured from cellulose. A strong collagen layer or a woven cellulose layer provides for a good positioning of the electrode on the fixation element. The cover layer also forms a friction-increasing surface for improved positioning and fixation of the fixation element.

It is advantageous if the cover layer forms a protective sleeve round a resilient element or an expandable body. Such a sleeve has a force-absorbing function. The electrode can be integrated with the cover layer. The cover layer can be adhered to the fixation element by means of adhesive means or by means of mechanical coupling, in particular by making use of known connecting techniques for textiles, such as sewing. A woven cover layer is preferably made from biodegradable and biocompatible fibres, wherein both natural and synthetic fibres can be envisaged.

The electrode preferably comprises at least one contact surface for exchanging electrical signals with a body organ, wherein at least a side remote from the contact surface is at least partially electrically shielded. This prevents electrical signals from and to the electrode being disrupted by the surrounding body organs. Such a shielding can be achieved by encasing the electrical conductor at least partly with a medically certified plastic such as polyurethane or polyethylene.

It is advantageous if the device is provided with feed means connected to the fixation element for feed of a fluid medium. A fluid medium such as a liquid, gas or foam can thus be fed to the fixation element. Such a medium can for instance be used to inflate an expandable body of the fixation element. Another possible application is the feed of medication, for instance inflammation inhibitors, which can be administered locally through the lumen and via the fixation element. The medication is hereby used more effectively and there are fewer side-effects because the used dosage of locally required medication can be reduced by such a local administering. The feed means can for instance take the form of a tube or lumen. The fixation element is preferably adapted to receive liquid and to release the liquid, preferably according to a slow-release principle.

In a preferred embodiment the feed means are at least partially integrated with the communication means of the electrode. It is thus particularly easy to introduce the feed means simultaneously with the communication means at the desired position into the body.

The invention further provides a method for manufacturing the device according to any of the foregoing claims, comprising the steps of: providing the electrode, a compressible resilient element and communication means, connecting the electrode and the resilient element, connecting the electrode and the communication means; and arranging fixing means on at least one side of the resilient element. The fixing means preferably comprise a biologically acceptable adhesive, preferably on the basis of gelatin, fibrinogen, collagen, chitin, chitosan or cellulose. The method preferably also comprises of arranging a protective, releasable film over a layer of adhesive. By arranging the film over the layer of fixing means this latter can be conditioned better during storage. If desired, the film can be kept on the layer of fixing means during introduction into the patient, with the advantage that the device displays no undesirable adhesion to surgical instruments or body parts until the film is removed.

In a preferred embodiment the method also comprises the step of coupling the device to surgical instruments in preparation for the introduction and placing of the electrode in a patient. Suitable surgical instruments are known to the skilled person and comprise for instance a catheter for guiding, among other parts, the electrode and electrical conductors connected to the resilient element placed under bias to the desired position in the body.

The invention also provides a method for treating a patient with a device according to the invention, comprising the method steps of introducing a device according to the invention as described above in a compact state of the fixation element, and expanding the fixation element until the fixation element is pressed under bias against the body organ to be treated. The fixation element is optionally further fixed by an adhesive arranged on the fixation element.

The invention further provides a kit of parts, comprising a device according to the invention and at least one surgical instrument adapted for introduction of the device, in particular a catheter. Using such a kit it is easy to introduce the device into a patient.

The invention will be elucidated on the basis of non-limitative exemplary embodiments shown in the following figures. Herein:

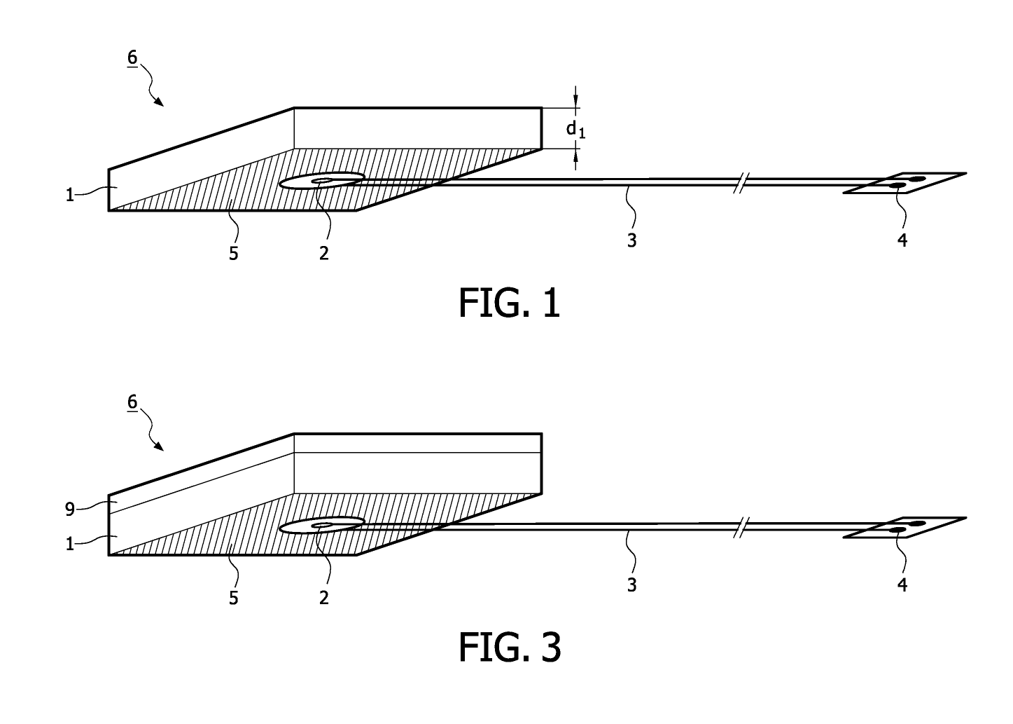

FIG. 1 shows a schematic perspective view of a device for in vivo fixing and positioning of electrodes according to the invention;

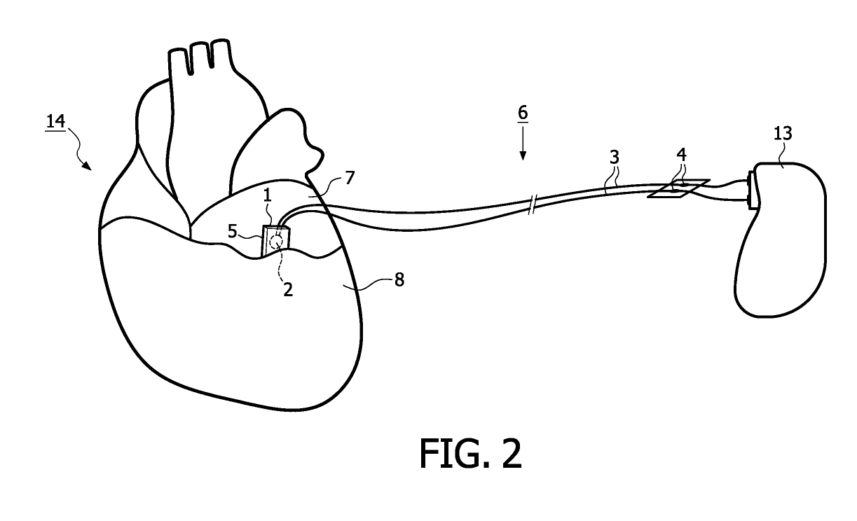

FIG. 2 shows a schematic perspective, partly cut-away view of a device for in vivo fixing and positioning of heart stimulating electrodes on at least a part of the heart according to the invention, and an activating member;

FIG. 3 shows a schematic perspective view of an alternative embodiment;

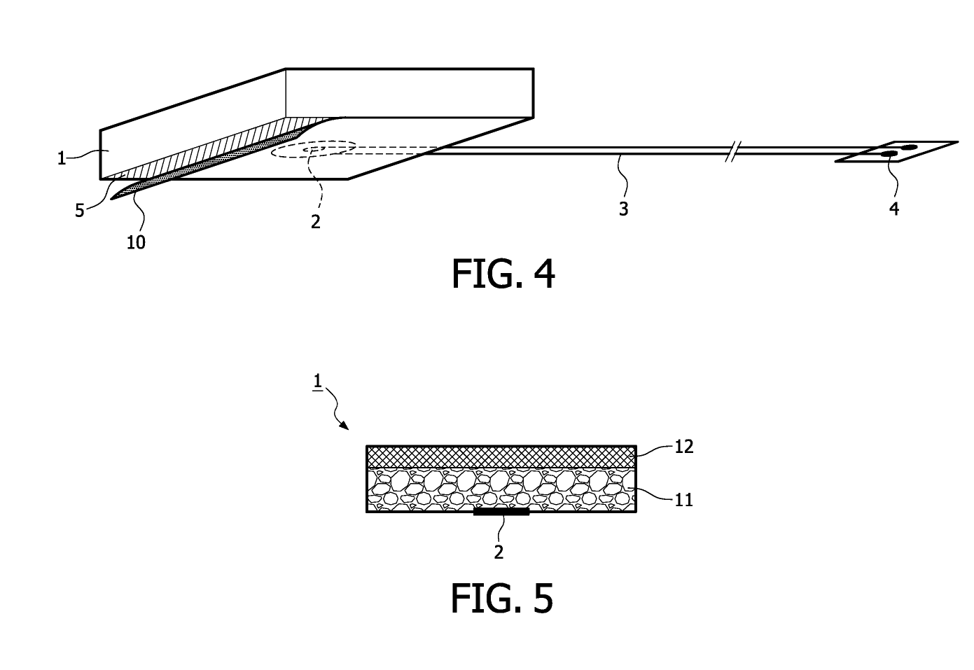

FIG. 4 shows a schematic, partly cut-away perspective view of another alternative embodiment;

FIG. 5 is a detailed cross-sectional view of an alternative embodiment of the embodiment shown in FIG. 3;

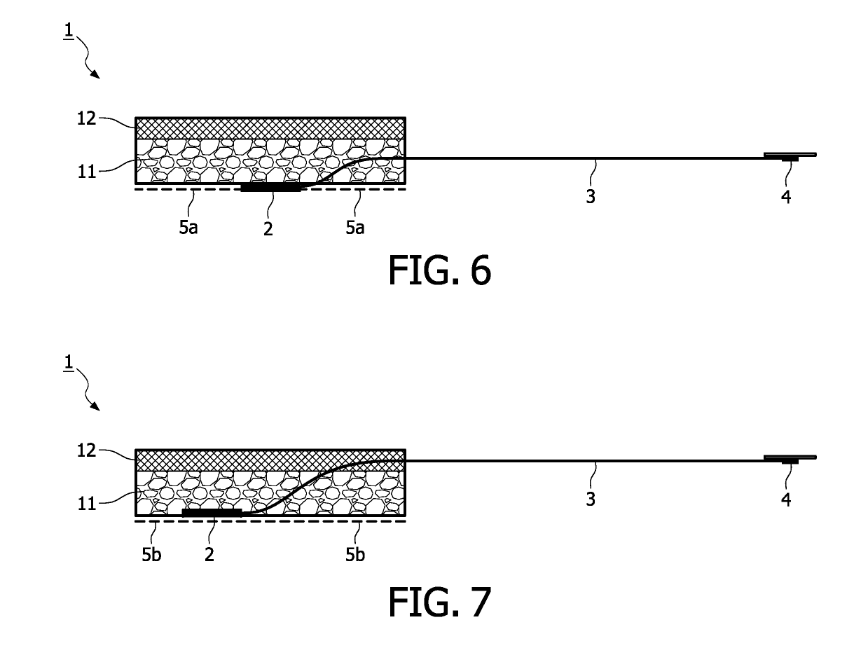

FIG. 6 is a detailed cross-sectional view of another alternative embodiment of the embodiment shown in FIG. 3;

FIG. 7 is a detailed cross-sectional view of yet another alternative embodiment of the embodiment shown in FIG. 3;

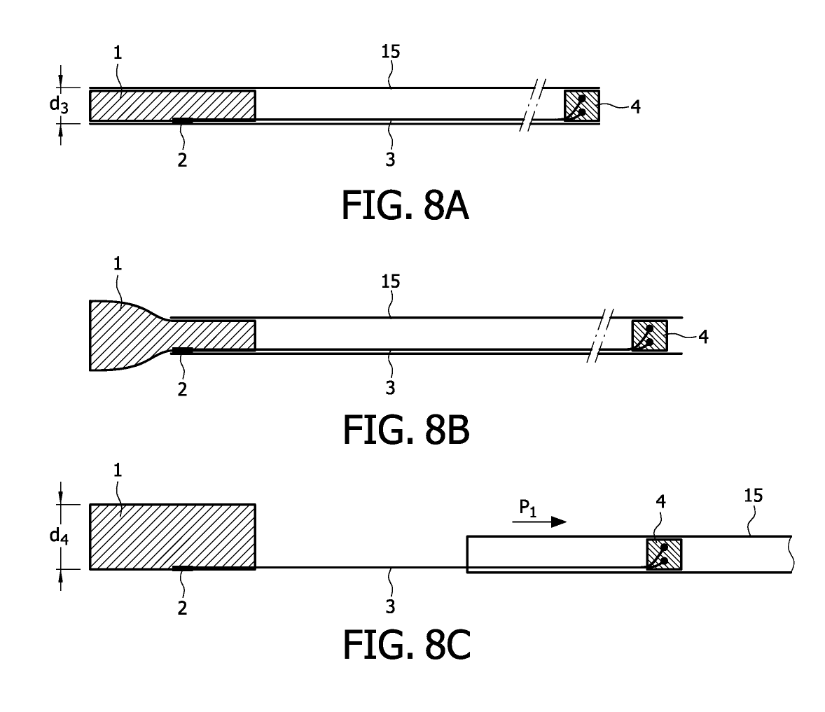

FIG. 8 shows detailed cross-sectional views of an embodiment of the device according to the invention, wherein in:

FIG. 8A the fixation element is fully enclosed by a catheter;

FIG. 8B the fixation element is partially enclosed by the catheter;

FIG. 8C the fixation element is situated outside the catheter;

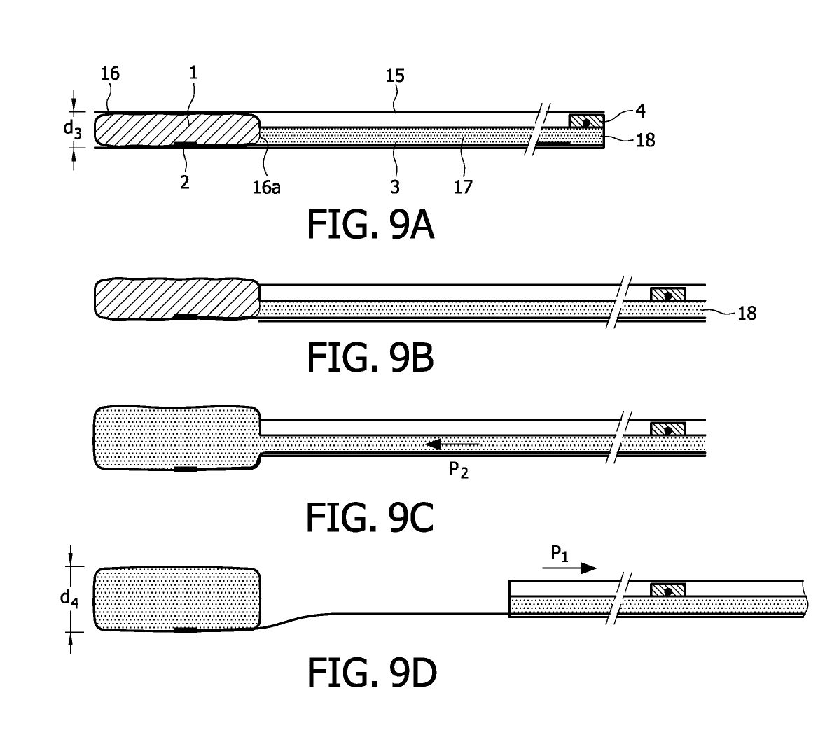

FIG. 9 shows detailed cross-sectional views of another embodiment of the device according to the invention, wherein in:

FIG. 9A the fixation element is fully enclosed by a catheter;

FIG. 9B the fixation element is situated outside the catheter;

FIG. 9C the fixation element is partially filled with a medium;

FIG. 9D the fixation element is wholly filled with a medium.

The same numbering is used in the different figures for comparable elements.

FIG. 1 shows a schematic perspective view of a device according to the invention, particularly suitable for application in treatment of a heart. The device is formed here by a resilient element 1 with an electrode 2 connected thereto, which electrode 2 is connected by means of electrical conductors 3 to electrical connecting clamps 4. The electrical conductors can for instance be manufactured from conductive metals. The underside of resilient element 1 is provided with fixing means 5. This device is designated as a whole with 6. Resilient element 1 here has a considerable thickness d₁ in non-biased state, wherein thickness d₁ is preferably between 2 and 25 millimetres for application in the case of a heart. It should be noted here that the thickness and other dimensions are generally many times smaller than the dimensions of the body organ for measuring and controlling, in particular the heart.

FIG. 2 shows a schematic, partly cut-away perspective view of a device according to the invention, wherein electrode element 2 comprises heart stimulating electrodes which are connected to at least a part of heart 14 and to an activating member 13. Features of heart 14 essential to the proper functioning of the device according to the invention are shown, including heart muscle 7 and pericardium 8, together with device 6 and activating member 13. Resilient element 1 is placed here between pericardium 8 and heart muscle 7, wherein pericardium 8 is a fibrous double-layered sac which encloses the heart and presses resilient element 1 against heart muscle 7. As a result the electrode 2 and fixing means 5 are also pressed against heart muscle 7. The thickness d₂ of resilient element 1, now in biased state caused by the pericardium, is smaller here than thickness d₁. By selecting a sufficiently great thickness of the resilient element the desired bias of the resilient element against the body organ is realized and a reliable fixation is brought about without the heart here being damaged, as happens with systems using barbs. Connected to connecting clamps 4 is a measuring and control member 13 for measuring and controlling the heart in co-action with the device according to the invention. Measuring and control member 13 can for instance be a pacemaker.

FIG. 3 shows an alternative embodiment wherein a cover layer 9 is arranged on resilient element 1.

FIG. 4 shows another embodiment, wherein a film 10 is arranged over the electrode 2 and fixing means 5 arranged on resilient element 1. The film is preferably made of a polymer, for instance a suitable nylon (polyamide) or Teflon® (polytetrafluoroethylene).

FIG. 5 is a detailed cross-section of an embodiment variant of the embodiment shown in FIG. 3, wherein the core of resilient element 1 is manufactured from sponge-like collagen 11 and cover layer 9 is manufactured from a woven cellulose 12. Sufficiently large dimensions are here also selected (preferably at least 15 mm×20 mm×5 mm) to enable a sufficiently reliable wedging of the resilient element between the targeted body organ and the surrounding tissue.

FIG. 6 is a detailed cross-section of an embodiment variant of the embodiment shown in FIG. 3, wherein electrode 2 is fixed in the interior of resilient element 1 with electrically conductive wires 3. The fixation can for instance be based on friction between the surfaces for fixing; it is however important here that the dimensions of the expanded state are sufficiently large. The embodiment shown in this figure also shows non-electrically conductive fixing means 5a, wherein it is therefore desirable that electrode 2 is in direct contact with the body organ for measuring and controlling.

FIG. 7 is a detailed cross-section of another embodiment variant of the embodiment shown in FIG. 3, wherein electrode 2 is fixed in cover layer 9 of resilient element 1 with electrically conductive wires 3. A fixing of the electrically conductive wires in the fabric is for instance created by interweaving the electrically conductive wires during weaving of the cover layer. It is also possible to fix electrode 2 and electrically conductive wires 3 in the interior of resilient element 1 as well as in cover layer 9. Electrically conductive fixing means 5b are also shown here, wherein electrode 2 does not need to make direct contact with the body organ for measuring and controlling. It is important that the resilient element is dimensioned for the cavities around the body organ for treating, such that conductive fixing means 5b engage with sufficient bias on the surface of the body organ for treating. A fixation element with the dimensions 30 mm×50 mm×20 mm is for instance particularly suitable for use with a heart.

FIG. 8A shows an embodiment as shown in FIG. 1, wherein fixation element 1 is resilient and enclosed by a catheter 15. Connected to fixation element 1 is an electrode 2, electrode 2 being connected to electrical connecting clamps 4 by means of the electrical conductors 3. In this situation the resilient fixation element 1 has a relatively compact form characterized by small thickness d₃, since it is placed under bias in catheter 15.

In FIG. 8B fixation element 1 of the embodiment as shown in FIG. 1 is partially enclosed by catheter 15, for instance because it has been partially retracted by a user. The thickness of resilient fixation element 1 in the situation as shown in FIG. 8B has increased locally relative to the situation as shown in FIG. 8A since the blockage imposed by catheter 15 has been removed. A part of the fixation element hereby expands and can hereby be fixed under bias between the surface of a body organ for treating and surrounding tissue.

In FIG. 8C fixation element 1 is situated wholly outside catheter 15 and has taken on the relatively voluminous form characterized by greater thickness d₄. When fixation element 1 is fixed at the correct position, catheter 15 can now be wholly withdrawn, as indicated by arrow P₁. Resilient fixation element 1, in combination with electrode 2, electrical conducting means 3 and electrical connecting clamps 4, is now situated at the correct position for fixing by wedging onto the surface of the body organ, or is already connected thereto.

FIG. 9A shows another embodiment wherein fixation element 1 comprises a balloon 16 and is enclosed by a catheter 15. Connected to fixation element 1 is an electrode 2, electrode 2 being connected to electrical connecting clamps 4 by means of electrical conductors 3. In this situation fixation element 1 has a relatively compact form characterized by the small thickness d₃ since balloon 16 is uninflated. Balloon 16 comprises an opening 16a with which balloon 16 is connected to a lumen 17 placed in catheter 15. A medium 18 with which balloon 16 is filled can be fed to balloon 16 through lumen 17.

In FIG. 9B fixation element 1 is situated with balloon 16 wholly outside catheter 15, for instance because it has been withdrawn by a user. The thickness of fixation element 1 has not necessarily increased relative to the situation as shown in FIG. 8A, since it does not need to be placed under appreciable bias in the catheter and is not yet filled with medium 18.

In FIG. 9C fixation element 1 is situated with balloon 16 wholly outside catheter 15, the same as the situation shown in FIG. 9B, and is still connected to lumen 17. Medium 18, for instance a foam manufactured substantially from gelatin, is now introduced into balloon 16 through opening 16a via lumen 17, whereby the volume of balloon 16 increases. The passage of medium 18 through lumen 17 to balloon 16 is indicated with arrow P₂. Balloon 16 of fixation element 1 is eventually wholly filled with medium 18 and herein takes on thickness d₄, this being shown in FIG. 9D. Medium 18 can for instance be a foam which has a first fluid state and a second solid state. Once the foam has taken on the solid state, catheter 15 can be released from balloon 16, for instance by a rotation round the longitudinal axis of catheter 15, wherein the foam in solid state will break under the torsional stress. Catheter 15 can now be retracted in its entirety, this being shown by arrow P₁. For a reliable fixation under sufficient bias the fixation element 1 must be dimensioned such that the intended space available between the surface of the body organ for treating and surrounding tissue is smaller than thickness d₄. The fixation element preferably expands such that the eventual volume without bias is between 25-100% greater than the compact volume with thickness d₃ of the fixation element in the catheter.

1-23. (canceled)

24. A device for in vivo positioning, repositioning and fixing of electrodes against body organs, in particular the human heart, comprising:
at least one electrode adapted to exchange electrical signals with a body organ,
communication means connected to the electrode for co-action with measuring and control means, and
at least one resilient fixation element connected to the electrode, wherein the fixation element is expandable from a relatively compact form to a relatively voluminous form, and
wherein the fixation element is adapted to press the electrode under bias against the body organ in the relatively voluminous form in co-action with surrounding body tissue in order to maintain the desired position.

25. The device as claimed in claim 24, wherein the fixation element comprises at least one resilient element.

26. The device as claimed in claim 24, wherein the fixation element comprises at least one expandable body.

27. The device as claimed in claim 26, wherein the expandable body is provided with feed means for feeding a swelling medium to the expandable body.

28. The device as claimed in claim 24, wherein the fixation element is provided with fixing means for adhering the fixation element to the body organ.

29. The device as claimed in claim 28, wherein the fixing means comprise a biologically acceptable adhesive.

30. The device as claimed in claim 29, wherein the adhesive is an adhesive which can be activated.

31. The device as claimed in claim 30, wherein the actuable adhesive can be activated by mechanical pressure.

32. The device as claimed in claim 28, wherein, at least a part of the fixing means is protected by a releasable film.

33. The device as claimed in claim 24, wherein the electrode is connected releasably to the fixation element.

34. The device as claimed in claim 33, wherein the electrode is connected to the fixation element such that the mechanical resistance for releasing the electrode from the fixation element is lower than the adhesion resistance between the fixation element and the body organ.

35. The device as claimed in claim 24, wherein the fixation element is manufactured substantially from a biodegradable material.

36. The device as claimed in claim 35, wherein the biodegradable material comprises at least one protein selected from the group consisting of gelatin, collagen and fibrinogen.

37. The device as claimed in claim 35, wherein the biodegradable material comprises at least one polysaccharide selected from the group consisting of cellulose, chitin, chitosan and carrageenan.

38. The device as claimed in claim 24, wherein the biodegradable material forms a resilient sponge structure.

39. The device as claimed in claim 38, wherein the biodegradable material is formed substantially from sponge-like collagen.

40. The device as claimed in claim 24, wherein the fixation element is at least partially provided with a reinforced cover layer.

41. The device as claimed in claim 40, wherein the cover layer is manufactured substantially from reinforced collagen.

42. The device as claimed in claim 24, wherein the electrode comprises at least one contact surface for exchanging electrical signals with a body organ, wherein at least a side remote from the contact surface is at least partially electrically shielded.

43. The device as claimed in claim 24, wherein the device is provided with feed means connected to the fixation element for feed of a fluid medium.

44. The device as claimed in claim 41, wherein the feed means are at least partially integrated with the communication means of the electrode.

45. A method for manufacturing a device for in vivo positioning, repositioning and fixing of electrodes against body organs, comprising the steps of:
providing an electrode, a compressible resilient element and communication means,
connecting the electrode and the resilient element,
connecting the electrode and the communication means;
arranging fixing means on at least one side of the resilient element.

46. A kit of parts, comprising a device as claimed in claim 24, and at least one surgical instrument adapted for introduction of the device, in particular a catheter.
